(19) United States
(12) Patent Application Publication
(10) Pub. No.: US 2008/0286346 A1
(43) Pub. Date: Nov. 20, 2008

(54) Wound Dressing

(76) Inventors: Rudolf Goerl, Obernburg (DE); Jochem Effing, Kelkheim-Fischbach (DE)

Correspondence Address: DREISS, FUHLENDORF, STEIMLE & BECKER, POSTFACH 10 37 62, D-70032 STUTTGART, DE

(21) Appl. No.: 11/630,349

(22) PCT Filed: Jun. 10, 2005
(86) PCT No.: PCT/EP05/06236

§ 371 Date: Dec. 21, 2006

(30) Foreign Application Priority Data
Jun. 24, 2004 (DE) 10 2004 031 955.3

(51) Int. Cl. A61K 9/70; A01N 59/16; A61P 17/02

(52) U.S. Cl. 424/445; 424/618

(57) ABSTRACT

A wound dressing comprises a support provided with a first side which is oriented towards the surface which is to be treated and a second opposite side, the support comprising a metal having an antimicrobial effect. A least the first side of the support has a preparation for localized therapy.

The invention concerns a wound dressing with a support having a first side facing a surface to be treated, and a second opposite side which is provided with an antimicrobially effective metal.

Wound dressings of this type are e.g. used for treating infected wounds or as a preventive wound infection protection measure. The treatment of infected or contaminated wounds is often medically problematic, in particular for wounds that heal only slowly and chronic wounds, the progress of healing being greatly delayed by bacterial accumulation. Bacterial accumulation is also a great risk for acute injuries. For this reason, many attempts have been made to remove microorganisms from bacterially contaminated and infected wounds or kill them and/or prevent bacterial accumulation. In addition to oral administration of antibiotics, the pathogenic microorganisms may also be removed by topical application of disinfectants or antibiotics. However, many antiseptics are cytotoxic. Moreover, pathogenic germs are often resistant to antibiotics.

For this reason, antimicrobially effective metals and oxidants, e.g. iodine tincture, have been conventionally used for a long time to treat infected wounds.

DE 199 58 458 A1 discloses e.g. a wound dressing with antimicrobial properties, wherein self-adhesive or non-self adhesive polymer materials which are used for wound treatment are combined with silver-containing zeolithes, which can be worked into the polymer materials. The zeolithes thereby act as ion exchangers, wherein small, defined amounts of silver ions are released in moist surroundings to thereby guarantee a long-lasting antibacterial effect.

DE 30 33 606 discloses use of an anti-adhesive, antimicrobial wound bandage on the basis of bandage material and pharmaceutical preparations, wherein an anti-adhesive or anti-adhesively prepared woven or non-woven material of natural or synthetic fibers is coated with an antimicrobial preparation, e.g. silver sulfadiazine ointment and is used for local therapy.

WO 01/60599 A1 discloses provision of a multi-layer laminate comprising a first layer of a polymer material and a second layer which is connected to the first layer, wherein the second layer contains a bacteriocidic metal, the metal preferably being silver. One disadvantage of this design is that the release of the metal in case of contact with the moist surroundings of the wound surface cannot be controlled and an excessive amount of metal could be prematurely dispensed which may cause health problems and require premature replacement of the bandage.

WO 2004/007595 A1 discloses a foamed material comprising an antimicrobial component which is dispensed in controlled amounts.

WO 2004/002384 A1 discloses a bandage material with antibacterial, antiviral and/or antifungicidal activity, wherein ionic silver is dispensed in controlled amounts into the wound secretion, the bandage material consisting of a hydrophilic, anionic polymer which is preferably bonded to silver via a polar or ionic bond.

WO 02/085387 A2 illustrates use of an antimicrobially effective metal in the form of nanocrystals.

Departing from prior art, it is the underlying purpose of the invention to provide an alternative wound dressing, wherein an antimicrobially effective metal is dispensed in controlled amounts. A further object of the invention is to provide a method for producing an antimicrobially effective wound dressing, in particular, for treating wounds that are infected with pathological germs.

This object is solved in accordance with the invention by a wound dressing comprising a support having a first side facing the surface to be treated and a second opposite side, and having an antimicrobially effective metal, wherein the support comprises, at least on its first side, a preparation for localized therapy.

In accordance with the invention, the support comprises a preparation at least on its first side, wherein the preparation is disposed either directly on the support provided with metal or a continuous or discontinuous metal layer is initially disposed on the first side, onto which the preparation is disposed. It may also be desired to apply the preparation on both sides, in particular, when the wound dressing is inserted into the wound. In this case, the metal is also advantageously provided on both sides or the knitted fabric surrounds it.

The preparation which may, in particular, be an ointment or a cream thereby acts as a mediator between the wound dressing provided with the metal and the wound of the patient. This reliably prevents direct contact with the wound dressing and, in particular, also adhesion between it and the wound. This ointment or cream may moreover have a nursing effect in the area of the peripheral skin of the wound. When the wound dressing contacts the wound via the preparation, the metal, e.g. silver, from the wound dressing is released via the wound secretion through mediation of the preparation and enters into the wound via the preparation. The amount of released metal can be regulated through selection of the preparation. Elementary silver may, in particular, be used as the metal. The metal may be coated on the wound dressing or the support or be impregnated into the support. In accordance with a first embodiment, it may be disposed on the first side of the support facing the wound.

Knitted polyamide supports which consist, in particular, of a nylon thread may preferably be used as supports.

In order to obtain good mediation via the preparation, the preparation itself may have a high water adoption capacity. The preparation advantageously contains no water. In this manner, pre-oxidation of the metal prior to use of the wound dressing can be prevented or at least reduced. The metal ions are released only when the wound dressing contacts the actual wound and liquid is present. It is thereby ensured that the wound dressing remains effective within a defined range e.g. during storage. Moreover, an increased amount of secretion from a wound increases the release of the antimicrobially effective metal which is advantageous in that wound secretion is a culture medium for all types of germs. In this case, an increased amount of the active medium is released.

The wound dressing may, in particular, release less than 500 μg/100 cm² of antimicrobial material in 24 hours at 37° C. in 100 ml water, in particular less than 400 μg/100 cm² in 24 h at 37° C. in 100 ml water and in particular less than 300 μg/100 cm² in 24 h at 37° C. in 100 ml water. The wound dressing may, in particular, release more than 2 μg/100 cm² in 24 h at 37° C. in 100 ml water.

Finally, the preparation may comprise at least one agent selected from the group of mono-, di- or triglycerides, fatty acidic ester, oligomers of glycerine, fatty alcohols, fatty acid ester, ethoxylated fatty alcohols, ethoxylated fatty acids, polyethoxylene derivatives or dimerised fatty acid esters or derivatives thereof. The preparation may, in particular, be a mixture comprising a mixture of different mono-, di- and triglycerides and/or fatty acid ester oligomers of glycerine and/or polyethylene glycolene. One preparation is particularly advantageous which comprises 1 to 80 weight % of mono-, di- and triglycerides, 1 to 80 weight % of fatty acid ester oligomer of glycerine and 1 to 30 weight % of polyethylene glycol.

In a further advantageous embodiment, a water-bonding agent of biopolymers selected from the group of alginates, carboxymethyl cellulose or chitosane may be added to the preparation. With particular preference, 1 to 30 weight % of water-bonding agents are added to the preparation.

In a further embodiment, the preparation may comprise dimerised fatty acid esters or derivatives thereof and a water-bonding agent. The preparation may thereby, in particular, comprise 1 to 99 weight % of dimerised fatty acid ester or derivatives thereof and 1 to 30 weight % of water-bonding agents.

Application of a compound, in particular, of an ointment or cream of an amount of at least 50 g/m² of support material, in particular at least 100 g/m² support material and preferentially 100 to 250 g/m² support material is particularly advantageous.

In accordance with a particularly preferred embodiment, the support may have several layers. In this case, only one of the layers of the support material has an antimicrobial metal which can be released through the wound dressing. Alternatively, antimicrobial metals of the same or different types may be provided in several layers.

Finally, the preparation may not be applied to the full surface, leaving out individual areas of the wound dressing, e.g. in order to apply adhesive for fixing the wound dressing to the skin of a patient. Alternatively, the wound dressing may be provided with the preparation on the full surface or on both sides, in particular, when the wound dressing is used for large wounds and surfaces to be treated or insertion into a wound is desired.

In a further embodiment, the inventive wound dressing, as a primary wound dressing, may be combined with conventional wound dressings as secondary wound dressings. Secondary wound dressings may be e.g. wound dressings of hydrophilic foams, e.g. hydrophilic polyurethane foams, wound dressings on an alginate basis e.g. fibrous alginates, wound dressings of hydrogels e.g. polyurethane ureas, hydrocolloidal wound dressings or gauze. In this combination, the secondary wound dressing is disposed on the second side of the inventive wound dressing. It has turned out to be particularly favorable to provide the second side of the inventive wound dressing with a foil bandage, in particular a breathable foil bandage. In one particularly preferred embodiment, the inventive wound dressing has a self-adhesive and breathable polyurethane foil bandage on its second side.

The invention also concerns a method for producing an antimicrobial wound dressing comprising the following steps: providing a support having an antimicrobially effective metal, and disposing a preparation, preferably an ointment or a cream, onto at least one side of the support. It may preferably be applied in the form of a layer or coating. The preparation may thereby correspond to the above-described preparation. The invention finally also concerns use of a wound dressing comprising a support having an antimicrobially effective metal and a preparation disposed thereon, in particular, of the above-described type for producing an agent for wound treatment.

The invention is explained in more detail below with reference to embodiments.

The following test method was used to determine the silver ion concentration:

The sample was prepared to determine the ion release rate of metallized support materials in water as described below. Bi-distilled water, 65% nitric acid (for analysis, company Merk) and diluted nitric acid (2 mol/l) produced from the 65% nitric acid were used as reagents.

The glass devices used are cleaned with warm, diluted nitric acid prior to use, rinsed with bi-distilled water and dried. A round disc of a diameter of 50 mm of the material to be investigated is punched out using a punching tool and is weighed (analysis scales, weighing accuracy d_{d}=0.1 mg) for use as a test sample. This test sample is flatly disposed onto the bottom of a conical flask (ground-in stopper, NS 29) and covered with 10 ml bi-distilled water (volumetric pipette 10 ml).

The flask is closed and left at 37° C. during the measuring time (24 hours). The extract is then decanted into a container of dark glass or plastic material and analyzed. If required, the extract is centrifuged to separate solid components. 10 ml extract 10 μl 65% nitric acid are added after decanting and/or centrifuging for stabilization. A chemical blank solution (without sample) is produced under the same conditions. The silver content is finally determined through atomic absorption spectroscopy in accordance with DIN 38406-E18 (E18).

In order to determine the antimicrobial activity, the following test method was used: It was determined in accordance with the standard method ASTM E 2180-01. The measurements were performed using a Ringer solution on Gram-positive germs (staphylococcus aureus) and Gram-negative germs (staphylococcus aureus). The higher the determined absolute value, the more active is the sample to be investigated.

The wound dressing is a knitted polyamide support fabric which is coated with elementary silver, and is produced from nylon 6 trilobal threads with an edge length of 50 μm. The raw material (without silvering) has a surface density of 25 to 30 g/m² and a thread fineness of dₜₑₓ=22/1. This material contains knitted threads of between 11,360 and 13,640 m per square meter. This means that the surface is between 1.7 m² and 2.04 m². The layer thickness of the silver on the support material is approximately 700 nm. Conventional means (light microscope) do not show any difference between the silvered and non-silvered product in the triangular side. The surface density of the silvered knitted fabric is 35+/−3 g/m². The amount of elementary silver is thereby approximately 130 mg/g knitted polyamide support fabric. The surface resistance is <2/cm² (typically between 0.55 and 0.95/cm²).

Silver-coated knitted fabrics of this type are distributed by the company Statex, Bremen under the trade name Shieldex® Tüll type Charmeuse and used as support material.

Different ointment substances were used as preparation which are described below:

Softisan 378 (company Sasol, Witten): This is a trade name for a triglyceride mixture of a mixture of saturated, even-numbered vegetal fatty acids (C8 to C18) of vegetal origin (caprylic/capric/staearic triglyeride), which is a product that is physiologically harmless and is compatible with the skin and mucous and can be used in water-free preparations.

Softisan 649 (company Sasol, Witten): wool wax substitute, fatty acid ester of oligomeric glycerine (mainly diglycerine) CTFA name: caprylic/capric/isostearic/adipic triglyceride).

PEG xxx (company Clariant, Offenbach): polyethylene glycol of different chain lengths, non-ionogenic compounds formed form ethylene oxide units MG=44 (average molecular weight); general formula HO—CH₂—(CH₂—O—CH₂)ₙ—CH₂—OH with n=4-70.

Lusplan (company Nippon Fine Chemicals, Osaka): dimerised fatty acid ester in accordance with the following illustration:

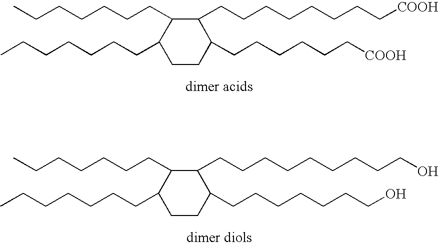

### Lusplan DD-DA7/DD-DA5

Polyesters of plant derived dimer diols and plant derived hydrogenated dimer acids.
INCI; dimer dilinoleyl dimer dillanoleate
Elincs; polymer

HO—R₁(—OCO—R₂—COO—R₁—)_{II}OH I=1.9
dimer acids are dibasic acids produced by the dimerization of C-18 unsaturated acids such as oleic acid or linoleic acid. We used hydrogenated dimer acid for oxidation stability.
dimer diols are saturated diols produced from dimer acids by the hydrogenation under high pressure and distillation.

Wound dressings with the most different preparations were produced, wherein the results of the tests are shown in the following table in relation to the silver ions discharged and the reduction in germination. The ointment substance has been applied in an amount of 185 g/m²±20% (relative to the support material coated with silver).

Example 1 is a support material with silvering, but without applied ointment substance. In example 2, the previously designated support material was used and a preparation consisting of 25% Softisan 649 and 75% Softisan 378 was disposed thereon. The samples 3 to 6 relate to coatings with PEG of different chain length. Sample no. 7 was coated with Lusplan 5. The ointment substances of samples 8 through 12 are mixtures of Softisan and PEG in different variants. In sample 13, Vaseline and tecero wax were added to Softisan 649 in amounts stated in the table. The examples 14 through 17 relate to products which are already on the market, wherein the product Antisorb® is a product of the company Johnson & Johnson and Acticoat® is a product by the company Smith & Nephew.

The amount of silver ions released was determined in accordance with the described method for 24 h and 37° C. for 100 cm² sample surface for all above-mentioned samples 1 through 17. The table shows values relating to μg/100 ml water or μg/ml water.

For a wound dressing which is not coated with the inventive preparation, ions are discharged in a range of 426.7 μg/100 ml water.

The maximum amount of silver ions was discharged for the product of the company Smith & Nephew Acticoat® with 9530.0 μg/100 ml water. This product is an antimicrobial wound bandage, in particular, against resistant microorganisms such as methicillin-resistant staphylococcus aureus (MRSA) or vancomycin-resistant enterococcus (VRE). The support material of the product Acticoat® is coated with nanocrystals.

The product Contreet H of the company Coloplast has a silver ion discharge of 1330 μg/100 ml. The product is a hydrocolloid bandage that uses ionic silver. With this product, a very large number of silver ions are also discharged, which is disadvantageous. The product by the company Convatec Aquasell AG, which is also an absorbent wound dressing, discharges a large amount of ionic silver which is considerably larger than the amount of silver discharged by the support material without coating, which is used in accordance with the invention with coating. In accordance with the invention, the amount of silver ions discharged should be less than 500 μg/100 cm² in 24 h at 37° C. in 100 ml water.

The product Actisorb® by the company Johnson & Johnson discharges silver ions of 40.6 μg/100 ml. This product is an activated charcoal compress which is surrounded by a non-woven material and is vapor-deposited with silver. This wound dressing is not treated with a preparation that ensures a defined discharge of silver ions.

The table shows that all inventive designs meet this criterion and discharge ions in an amount of, in particular, less than 350 mg/100 cm². The reduction in germs in accordance with the described test method was moreover determined for a selection. Reduction in germs was determined once after 4 and after 24 hours, which is shown in the conventional logarithmic form. The determined values for germ reduction of e.g. Gram-positive germs from log 1.9 after 4 hours or of 4.2 after 24 hours for example 2, and log 1.6 after 4 hours or log 4.7 after 24 hours for example 12, show that these examples have a good antimicrobial effect. Even after 24 hours, the germ reduction of log 4.6 is similarly high for a support material without coating with an ointment substance. This shows that relatively small silver discharge amounts having less toxic effect on the patient also achieve good germ reduction. Moreover, silver ions may be discharged over a longer time period by adding the ointment substance. Silver ions are thereby discharged in the desired controlled form after adding liquid through releasing ions.

| | | Silver ion discharge | | Germ | | | |
|---|---|---|---|---|---|---|---|
| | | for 24 h, 37° C., | | reduction ASTM | | Germ reduction | |
| | Support material | 100 cm² | | 2180 Gram- | | ASTM 2180 | |
| | coated with | sample surface | | positive | | Gram-negative | |
| | ointment substance | μg/100 ml | μg/ml | log10 in | log10 in | log10 in | log10 in |
| No. | consisting of: | water | water | 24 h | 24 h | 4 h | 24 h |
| 1 | Without coating | 426.7 | 4.2667 | 3.9 | 4.6 | 4.7 | 4.7 |
| 2 | 25% Softisan 649, | 9.1 | 0.0907 | 1.9 | 4.2 | — | — |
| | 75% Softisan 378 | | | | | | |
| 3 | 100% PEG 400, | 218.7 | 2.1867 | | | | |
| | (number of units, | | | | | | |
| | n = 9) | | | | | | |
| 4 | 100% PEG 600 | 186.7 | 1.8667 | | | | |
| | (number of units | | | | | | |
| | n = 14) | | | | | | |
| 5 | 100% PEG 1000 | 256.0 | 2.5600 | | | | |
| | (number of units | | | | | | |
| | n = 23) | | | | | | |
| 6 | 100% PEG 2000 | 314.7 | 3.1467 | | | | |
| | (number of units | | | | | | |
| | n = 46) | | | | | | |
| 7 | 100% Lusplan DD- | 80.0 | 0.8000 | | | | |
| | DA5 | | | | | | |
| 8 | 90% Softisan 649, | 5.3 | 0.0533 | | | | |
| | 10% PEG 1000 | | | | | | |
| 9 | 90% Softisan 649, | 16.5 | 0.1653 | | | | |
| | 10% PEG 2000 | | | | | | |
| 11 | 75% Softisan 649, | 4.8 | 0.0480 | | | | |
| | 25% PEG 400 | | | | | | |
| 10 | 80% Softisan 649, | 42.7 | 0.4267 | | | | |
| | 10% Softisan 378, | | | | | | |
| | 10% PEG 1000 | | | | | | |
| 12 | 25% Softisan 649, | 43.7 | 0.4373 | 1.6 | 4.7 | 4.9 | 4.9 |
| | 65% Softisan 378, | | | | | | |
| | 10% PEG 2000 | | | | | | |
| 13 | 68.2% Vaseline, | <2 | | 0.3* | 0.5* | | |
| | 29.8% Softisan | | | | | | |
| | 649, 2% Tecero | | | | | | |
| | wax | | | | | | |
| 14 | J&J Actisorb | 40.6 | 0.4060 | 2.2 | 4.7 | | |
| 15 | S&N Acticoat | 9530.0 | 95.3 | | | | |
| 16 | Coloplast, Contreet | 1330 | 13.3 | | | | |
| | H | | | | | | |
| 17 | Convatec, Aquadell | 664 | 6.64 | | | | |
| | AG | | | | | | |

*according to method AATCC 100-1999

The invention is explained below with reference to the drawing.

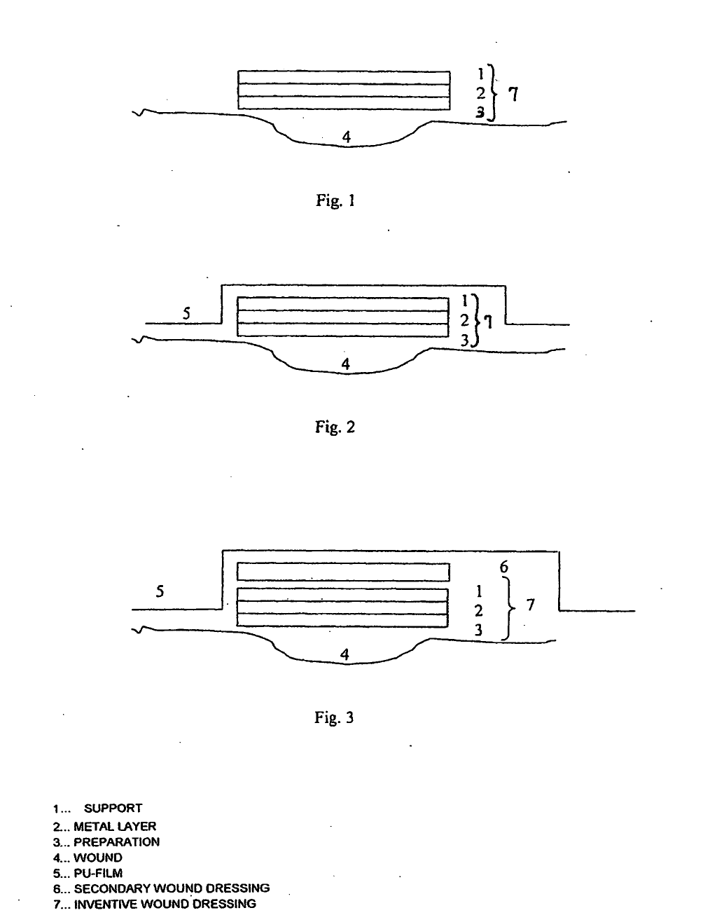

FIG. 1 shows a first embodiment of a wound dressing;

FIG. 2 shows a further embodiment of a wound dressing;

FIG. 3 shows a third embodiment of a wound dressing.

The first figure shows a first embodiment of an inventive wound dressing comprising a support 1 of a knitted nylon fabric. A metallic coating 2 is provided on the support 1 on its side facing the wound 4 to be treated, which has an antimicrobial effect and consists of elementary silver in this case. A preparation 3 in the form of an ointment or cream is also provided on the metal-coated side of the support 1, in particular spread or coated. The wound 4 is thereby only directly in contact with the preparation 3 which controls discharge of metal ions from the metallic coating 2 by mediating contact between liquid and the metal layer 3. Such a wound dressing may either be fixed to the wound 4 using e.g. a bandage or be inserted directly into a wound 4.

FIG. 2 shows a wound dressing which corresponds substantially to the wound dressing 7 (also primary wound dressing 7) in accordance with FIG. 1. Identical parts have identical reference numerals. In addition to the primary wound dressing 7 in accordance with FIG. 1, a film 5 (polyurethane film) is also provided which is applied on the side of the primary wound dressing 7 facing away from the wound 4, and covers it completely when applied onto the skin of a patient. The film 5 is thereby connected, in particular glued, to the primary wound dressing 7 in a material-bonding connection.

The film 5 moreover has an adhesive coating in its edge areas which project past the primary wound dressing 7, such that the wound dressing 7 can be fastened to the skin of a patient. The adhesive coating may thereby be a continuous or discontinuous coating which is conventional for adhesive wound dressings. The substantial parameters are thereby skin compatibility (water vapor permeability, optionally water density, breathability, anti-allergic properties, good releasability) and also adhesive force (secure retention). The dimensions of the wound dressing 7 are thereby selected such that the adhesive coating rests on skin that is not injured.

FIG. 3 shows a further design, wherein the same parts also have the same reference numerals. In addition to the primary wound dressing 7, a secondary wound dressing 6 is provided which is disposed on a second side of the support 1 facing away from the wound 4 and meets additional tasks for wound treatment such as in particular cushioning and liquid absorption.

As in FIG. 2, a polyurethane film is provided on the side of the secondary wound dressing facing away from the wound 4 to cover the entire wound dressing, comprising the primary and secondary wound dressing, and covers it from the surroundings when applied.

1-20. (canceled)

21. A wound dressing for a surface to be treated, the dressing comprising:
a support having a first side facing the surface to be treated;
an antimicrobially effective metal; and
a preparation for localized treatment, said preparation disposed between said first side of said support and the surface to be treated.

22. A wound dressing for a surface to be treated, the dressing comprising:
a support having a first side facing the surface to be treated;
an antimicrobially effective metal; and
a preparation for localized treatment, said preparation disposed between said first side of said support and the surface to be treated,
wherein said preparation is an ointment or a cream.

23. The wound dressing of claim 21, wherein said preparation contains no water.

24. The wound dressing of claim 21, wherein said metal is applied to said support as a coating or is impregnated onto or into said support.

25. A wound dressing for a surface to be treated, the dressing comprising:
a support having a first side facing the surface to be treated;
an antimicrobially effective metal; and
a preparation for localized treatment, said preparation disposed between said first side of said support and the surface to be treated,
wherein said antimicrobially effective metal is disposed on said first side of said support.

26. The wound dressing of claim 21, wherein said metal is silver.

27. The wound dressing of claim 21, wherein said support is a knitted polyamide fabric or a knitted nylon fabric.

28. The wound dressing of claim 21, wherein said preparation is coated or spread on said first side of said support.

29. The wound dressing of claim 21, wherein the wound dressing releases less than 500 μg/100 cm² of antimicrobial metal in 24 h at 37° C. in 100 ml water.

30. The wound dressing of claim 29, wherein the wound dressing releases less than 400 μg/100 cm² of antimicrobial metal in 24 h at 37° C. in 100 ml water.

31. The wound dressing of claim 21, wherein the wound dressing releases at least 2 μg/100 cm² of antimicrobial metal in 24 h at 37° C. in 100 ml water and less than 300 μg/100 cm² in 24 h at 37° C. in 100 ml water.

32. The wound dressing of claim 21, wherein said preparation comprises at least one agent selected from the group consisting of mono-, di- or triglycerides, fatty acid ester oligomer of glycerine, fatty alcohols, fatty acid ester, ethoxylated fatty alcohols, ethoxylated fatty acids, polyethoxylene derivatives or dimerised fatty acid esters or derivatives thereof.

33. The wound dressing of claim 32, wherein said preparation comprises at least one water-bonding agent.

34. The wound dressing of claim 32, wherein said preparation comprises a mixture of mono-, di- and triglycerides and/or fatty acid ester oligomer of glycerine.

35. The wound dressing of claim 32, wherein said preparation comprises at least one water-bonding agent and dimerised fatty acid ester.

36. The wound dressing of claim 21,
wherein said the support has several layers.

37. The wound dressing of claim 21, wherein said preparation is not applied to a full surface.

38. A method for producing the antimicrobial wound dressing of claim 21, the method comprising the steps of:
a) providing a support coated with an antimicrobially effective metal; and
b) applying a preparation for local therapy an ointment or cream onto at least one side of the support.

39. The method of claim 38, wherein said preparation is coated or spread onto said support.

40. Use of the wound dressing of claim 21 to generate means for treatment of wounds.
